(19) United States
(12) Patent Application Publication
(10) Pub. No.: US 2013/0317445 A1
(43) Pub. Date: Nov. 28, 2013

(54) SHIELD APPARATUS

(71) Applicant: Graham Steer, London (GB)

(72) Inventor: Graham Steer, London (GB)

(73) Assignee: PROSYS INTERNATIONAL LTD, London (GB)

(21) Appl. No.: 13/981,860

(22) PCT Filed: Jan. 27, 2012
(86) PCT No.: PCT/EP12/51365

§ 371 Date: Aug. 12, 2013

(30) Foreign Application Priority Data
Jan. 27, 2011 (GB) 1101402.4
Feb. 16, 2011 (GB) 1102690.3

(51) Int. Cl. A61M 25/02

(52) U.S. Cl. CPC A61M 25/02
USPC 604/180

(57) ABSTRACT

Apparatus (10) for shielding a catheter from contact with water whilst showering, the apparatus (10) comprising: a first shield component (10(a)) having a first part (13) that is configured to form a first seal around an exit aperture (30) from which a catheter exits the patient's body, said first part (13) defining an internal volume (17) that opens to an internal volume (15) of a second part (15) that is configured to receive a distal part of said catheter; and a second shield component (25) for adhering to the patient's body around at least part of the periphery of the first part (13) of said first shield component (10(a)) so as to provide a second seal against water contact with said exit aperture (30); wherein said first and second shield components (10(a), 10(b)) cooperate, when the apparatus (10) is worn by a patient, to resist water contact with said exit aperture (30) without obstructing access to an area of the patient's skin beneath said second part (15) of said first shield component (11).

### FIELD

The present invention relates to apparatus that is configured to be worn by a person to whom a catheter (such as a central venous catheter) has been fitted. In one illustrative application, use of such an apparatus reduces the likelihood of water coming into contact with a person's catheter while they shower.

### BACKGROUND

Central venous catheters (CVCs) are configured to be in direct communication with the bloodstream of a patient. As such, the risk of infection and blood poisoning to a person wearing a CVC is significantly increased. Alarmingly, the article “Keeping Central Line Infections at Bay” Nursing 36(4):58-64, April 2006 by L. Hadayaw reports that the mortality rate for CVC related bloodstream infections is estimated to be between 4% and 20%. Blood poisoning is typically caused by substances and foreign bodies coming into contact with the site in a person's body through which a CVC protrudes (typically referred to as a catheter exit site). One such substance that could carry pathogens and/or bacteria into a CVC exit site is water, and because of this it is typically the case that patients to whom a CVC has been fitted are usually instructed not to take a shower or a bath.

Clearly the inability for a patient to take a shower or a bath causes disruption to their lifestyle as they are restricted to using less efficient methods of cleaning themselves. This problem is particularly acute for those patients, such as kidney dialysis patients, who are often fitted with a catheter for an extended period of time.

One previously proposed device that seeks to address this problem is disclosed in US Patent Application No. US2008/0208145. This document describes a disposable guard that a patient with a CVC wears around their catheter to enable them to take a shower. With reference to FIG. 1, such a guard 1 comprises a bag 2 into which the distal end of a catheter 4 is inserted. The bag 2 is then adhered to a person's body 6 around the exit site through which the catheter protrudes, thereby sealing the catheter 4 within the bag 2.

Whilst this arrangement does provide some measure of protection, problems may occur if the bag has not been properly sealed to the skin by the patient. For example, if there are hairs trapped between the bag 2 and the patient's skin 6 then there is the possibility that water may be able to seep through the seal and into the bag 2. Unfortunately, this problem is exacerbated by the fact that the bag design tends to cause water to pool between the top of the bag and the skin (i.e. in the region indicated by reference numeral 3) when a patient is standing in a shower.

Another device that seeks to address similar problems is disclosed in European Patent No. EP1420848. The device disclosed in this document also includes a bag into which the distal end of a catheter is inserted. The bag is then adhered to a patient's body around the entrance site of their catheter thus forming a first barrier for restricting water ingress into the bag. A flexible shield is then adhered to the patient's body around the aforementioned bag so that it completely encloses the bag and thereby forms a second barrier to water ingress.

Whilst this arrangement further reduces the chance of water entering the bag through the seal, the shield is necessarily quite large and as a consequence it can be difficult for a patient to manipulate the shield into position without assistance. This problem is exacerbated in cases where the CVC in their neck or upper chest, as part of the shield will extend over the shoulder (as shown in FIG. 2) or behind the head. A further problem associated with this arrangement is that as the shield is quite large (in any event larger than the bag into which the distal end of the catheter extends), it prevents the patient from washing a significant amount of their skin.

Aspects of the present invention have been devised with the foregoing problems in mind.

### SUMMARY

In accordance with a presently preferred embodiment of the present invention, there is provided apparatus for shielding a catheter from contact with water whilst showering, the apparatus comprising: a first shield component having a first part that is configured to form a first seal around an exit aperture from which a catheter exits the patient's body, said first part defining an internal volume that opens to an internal volume of a second part that is configured to receive a distal part of said catheter; and a second shield component for adhering to the patient's body around at least part of the periphery of the first part of said first shield component so as to provide a second seal against water contact with said exit aperture; wherein said first and second shield components cooperate, when the apparatus is worn by a patient, to resist water contact with said exit aperture without obstructing access to an area of the patient's skin beneath said second part of said first shield component.

An advantage of this arrangement is that the apparatus covers a much smaller proportion of the patient's skin than the apparatus depicted in FIG. 2, and hence the patient is more easily able to affix the apparatus to their body without assistance. A further advantage of this arrangement is that as the catheter tails are wholly enclosed in the second portion of the first component and the second component does not overly that second portion, so a user wearing the apparatus whilst showering can lift the second portion and wash the skin that lies beneath that part first component. Hence a user is prevented from washing a far smaller area of their skin than they are with the device depicted in FIG. 2, for example. A further advantage of this arrangement is that water cannot pool between the first component and the patient's skin.

In a preferred arrangement the second shield component is configured to at least partially cover the first part of said first shield component.

The second shield component may be provided with adhesive for forming said second seal. The adhesive provided on said second shield component may extend along at least one peripheral side of said second shield component. In another arrangement, the adhesive may extend along more than one peripheral side of said second shield component. In one envisaged arrangement, the adhesive provided on said second shield component is generally C-shaped.

In a preferred arrangement the first seal is located wholly within the C-shaped adhesive on said second shield component when the second component is fitted over the first shield component.

The first and second shield components may be capable of being coupled to one another. The first shield component and/or the second shield component may further comprise an additional body of adhesive for fixably coupling the first and second shield components together.

The first part of said first shield preferably comprises an entry aperture for receiving said catheter. The first shield component may comprise a liner accommodated within said aperture.

In one arrangement, substantially all of one face of said second component is covered with adhesive.

The second part of said first component may be narrower than said first part of said first component.

The second shield component may comprise a plurality of discrete cover components. In one particular arrangement the second shield component comprises first, second and third discrete cover components.

Such first, second and third cover components may each be configured to be coupled to the body of a patient and to the first part of the first shield component.

The first, second and third shield components may be configured to extend along respective edges of the first part of the first shield component when coupled thereto.

The third cover component may be configured to partially cover the first cover component and the second cover component when the first, second and third cover components are coupled to the first part of the first shield component.

Other features, aspects and advantages of the invention will be apparent from the following detailed description.

### BRIEF DESCRIPTION OF THE DRAWINGS

Various aspects of the teachings of the present invention, and arrangements embodying those teachings, will hereafter be described by way of illustrative example with reference to the accompanying drawings, in which:

FIGS. 1 and 2 each show a prior art CVC shield device;

FIG. 3 is a schematic plan view of apparatus according to a first embodiment of the invention;

FIG. 4 is a schematic plan view of the apparatus shown in FIG. 3 assembled in use;

FIG. 5 is a schematic plan view of apparatus according to a second embodiment of the invention;

FIG. 6 is a schematic plan view of apparatus according to a third embodiment of the invention;

FIGS. 7 and 8 are schematic plan and sectional views, respectively, of a shield for use with any of the aforementioned embodiments; and

FIGS. 9 to 11 are schematic plan views of apparatus according to a fourth embodiment of the invention.

### DETAILED DESCRIPTION

Illustrative implementations of the teachings of the present invention will now be described with particular reference to shield apparatus for a central venous catheter (CVC) that typically has two or more lumens. However, it should be remembered that this particular implementation is merely illustrative, and that the teachings of the present invention may equally be applied to a shield apparatus for use with any other type of catheter.

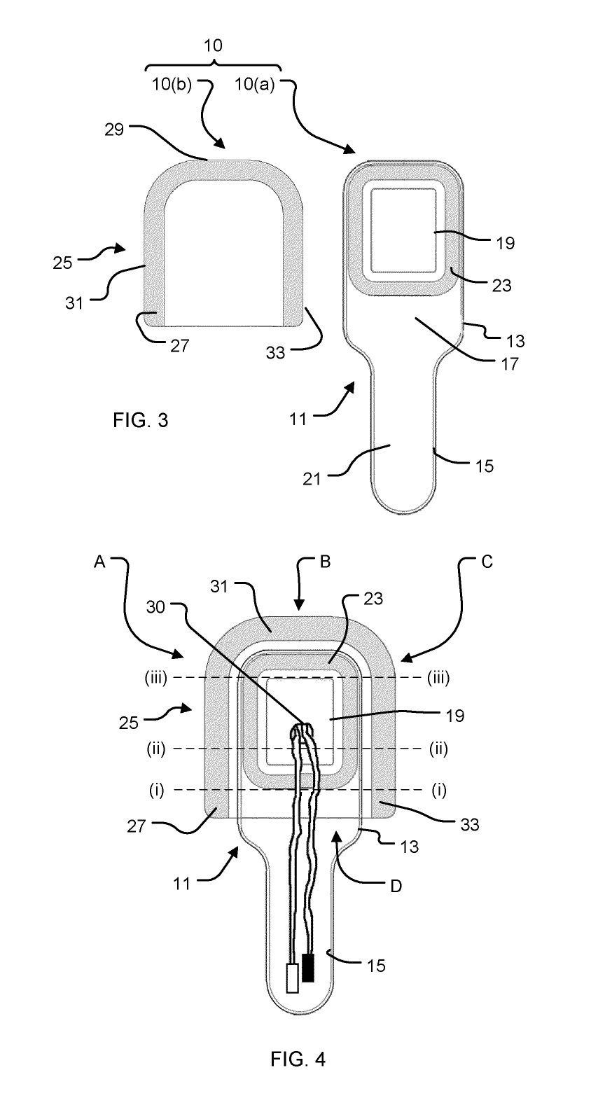

With the above proviso in mind, reference will now be made to FIGS. 3 to 5 of the accompanying drawings in which there is depicted a shield apparatus 10 that embodies the teachings of the invention. The shield apparatus 10 is configured to be worn by a patient to whom a CVC catheter has been fitted, and functions to reduce the likelihood of shower water entering the body via the catheter exit site.

With particular reference to FIG. 3 the shield apparatus 10 comprises first 10(a) and second 10(b) shield components that cooperate, in a manner that is later described in detail, to reduce the likelihood of water from a shower coming into contact with the exit site of a catheter.

The first shield component 10(a) is generally in the form of a bag 11 that functions to receive, envelop and thereby protect a distal portion of a catheter (not shown) that protrudes from a user. In an envisaged arrangement the bag 11 comprises a first portion 13 and a second portion 15.

The first portion 13 defines an internal volume 17 that can be accessed via an entry aperture 19, and which opens to an internal volume 21 of the second portion 15. The internal volume 21 of the second portion 15 receives the distal end(s) of one or more lumens of a catheter when the apparatus is worn, and in a preferred implementation (shown in FIG. 3) the first portion 13 may be wider than the second portion 15 to facilitate insertion of the catheter distal ends into the internal volume 21 of the second portion 15. As will be appreciated by persons skilled in the art, when the distal ends are located in the internal volume of the second portion, they are protected by the second portion from contact with water.

The bag 11 is provided with an adhesive strip 23 that preferably extends substantially all of the way round the periphery of the entry aperture 19 so that a user can couple the bag 11 to their body in such a way that the first portion 13 overlies the catheter exit site, and the adhesive strip 23 forms a seal around the exit site. The adhesive strip is preferably provided with a cover (for example of waxed paper) that a user removes to expose the adhesive.

The second shield component comprises a guard 25 that includes an adhesive strip 27 which extends at least partway around the periphery of the guard. In a particularly preferred arrangement, the adhesive strip extends along a first side 29 of the guard, which first side 29 is uppermost when the apparatus is worn by a user. The adhesive strip may also extend from the first side at least partway down second and third parallel sides 31, 33 (which sides will be generally vertical when the guard is adhered to a patient with the first edge uppermost).

The guard 25 is shaped and configured to fit over at least part of the first portion 13 of the bag 11, and when so arranged the adhesive strip 27 of the guard 25 provides a second seal against water ingress from those directions (namely sideways and downwards) in which water is most likely to be to be flowing whilst a patient takes a shower).

FIG. 4 is a schematic plan view of the two components of the shield apparatus assembled in use with the guard 25 overlying part of the first portion 13 of the bag 11. As will be appreciated from FIG. 4, the apparatus provides two seals against sideways water ingress (i.e in directions A & C), and downwards water ingress (i.e. in direction B). The apparatus only provides a single seal against water ingress in an upwards direction (i.e. in direction D), but as water flows downwards from a shower head, it is anticipated that a single seal will be more than adequate to deal with any water that might inadvertently splash upwards whilst the person fitted with the catheter is showering.

In a particularly preferred arrangement, as shown in FIG. 4, the adhesive strip 27 of the guard 25 is generally U-shaped and extends down the second and third guard sides 31, 33 to a point below the seal formed by the adhesive strip 23 on the first component. In this configuration the adhesive strip 23 on the first component is wholly within the U-shaped adhesive strip 27 on the guard 25.

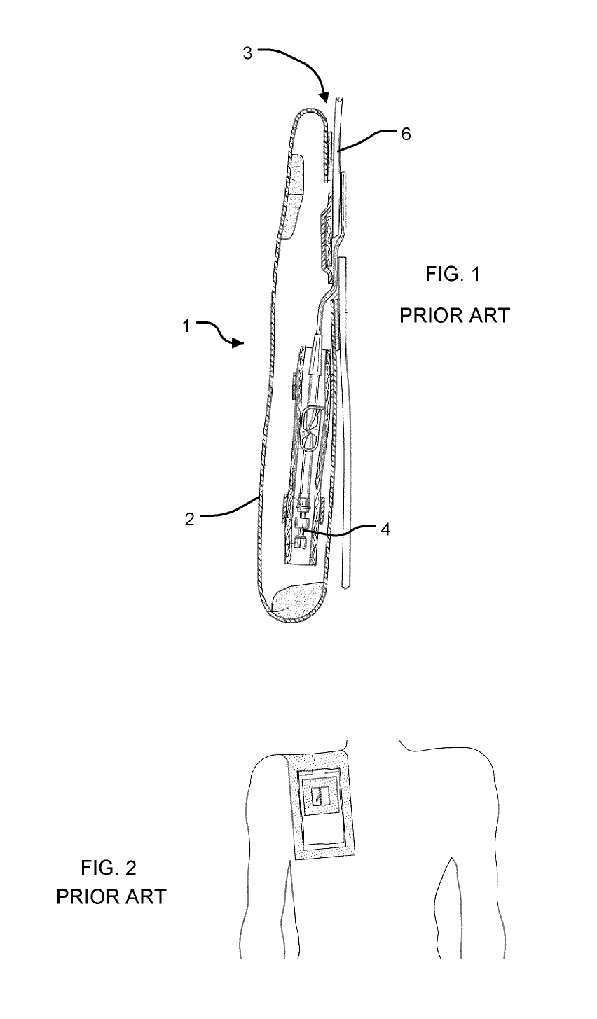

Whilst this arrangement is preferred, it will be appreciated that the adhesive strip 27 on the guard sides 31, 33 need not extend downwardly beyond the adhesive strip 23 on the first component, and could instead terminate at a higher point (i) in the vicinity of the adhesive strip 23 on the first component. Whilst it is particularly preferred for the adhesive strip 27 to at least extend to a point (ii) below the exit site 30, it will be appreciated by persons skilled in the art that an adhesive strip which terminated at or around point (iii) would still provide an arrangement that avoided many of the problems (in particular the aforementioned pooling problem) associated with the devices depicted in FIGS. 1 and 2 of the drawings.

As a further means of avoiding problems associated with pooling of water it is particularly preferred for the adhesive strip 27 of at least the guard to extend inwardly from the peripheral edge(s) of the guard. The adhesive strip 23 of the bag 11 may likewise extend inwardly from the peripheral edges of the bag 11 towards the aperture 19, or may instead be spaced therefrom.

As can easily be appreciated by comparing FIGS. 2 and 4, the apparatus depicted in FIG. 4 covers a much smaller proportion of the patient's skin than the apparatus depicted in FIG. 2, and hence the patient is more easily able to affix the apparatus to their body without assistance. A further advantage of the apparatus depicted in FIG. 4 is that as the catheter tails are wholly enclosed in the second portion of the bag and the guard 25 does not overly that second portion, so a user wearing the apparatus whilst showering can lift the second portion and wash the skin that lies beneath that part of the bag 11. Hence a user is prevented from washing a far smaller area of their skin than they are with the device depicted in FIG. 2, for example. A further advantage of the arrangement shown in FIG. 4 is that water cannot pool between the bag 11 and the adhesive strip 23 on the bag 11.

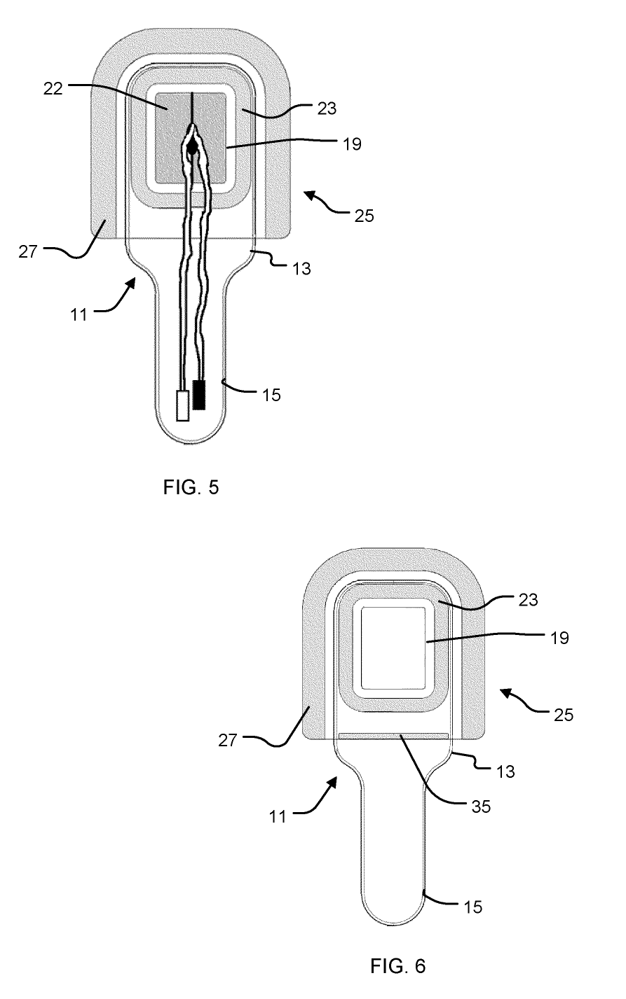

In the arrangement depicted in FIG. 4, the entry aperture 19 simply comprises a hole in the bag 11, but in a modification of this arrangement (shown in FIG. 5) the hole 19 in the bag 11 may be filled with an absorbent liner 22. Such a liner 22 may comprise a body of absorbent material with an opening (such as a slit) in the liner through which a user's catheter line extends when the catheter is inserted into the bag 11. As will be appreciated, the liner 22 provides a final line of protection to a user's catheter exit site by absorbing water that breaches the adhesive seal 23 provided on the bag 11. In another envisaged arrangement, the absorbent liner 22 may be coated and/or impregnated with steriliser for sterilising a user's catheter entrance site (such as an appropriate antiseptic or antibacterial substance for example).

In a further modification, depicted schematically in FIG. 6, the guard 25 may comprise a further strip of adhesive 35 that couples the guard 25 to the bag 11 when the guard 25 is positioned over the first part of the bag. In one envisaged arrangement the additional strip of adhesive may be located, as shown in FIG. 6, proximate a fourth edge of the guard 25 that is generally parallel to the first edge 29. As will be appreciated, the guard shown in FIG. 6 could be used with a bag of the type depicted in FIG. 5, and in a further modification the additional strip of adhesive could be provided on the bag 11.

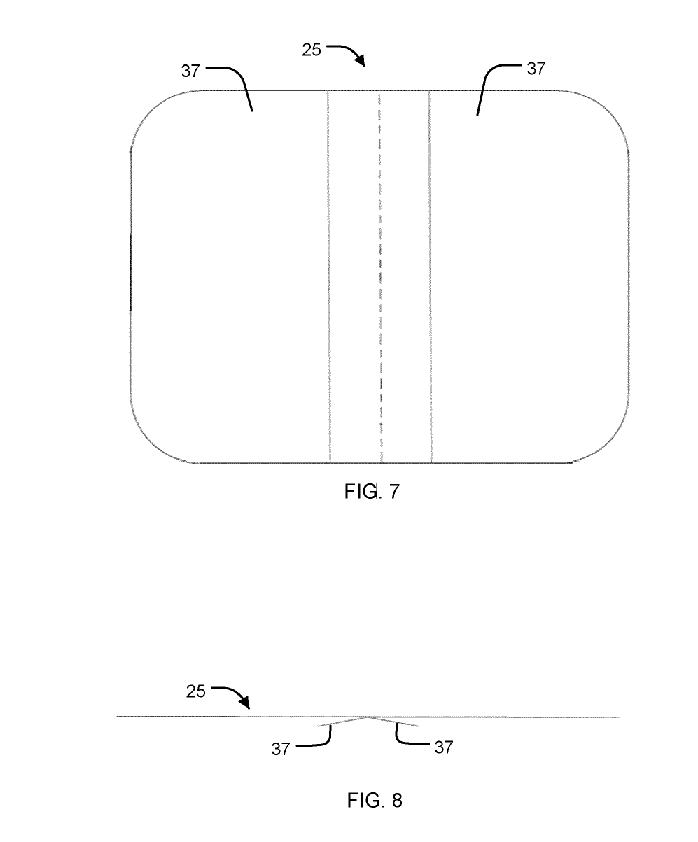

In yet another envisaged arrangement, substantially the entirety of the underside of the guard 25 may be provided with an adhesive so that the guard adheres to the skin and to the bag 11. In this arrangement the adhesive underside of the guard 25 may be provided, as shown in FIGS. 7 and 8, with two waxed adhesive covers 37 that can be removed by a user to expose the adhesive.

In a preferred implementation the bag 11 and guard 25 are formed of flexible, water impervious material. For example the bag 11 and guard 25 may be formed of polyethylene.

It will be appreciated that whilst various aspects and embodiments of the present invention have heretofore been described, the scope of the present invention is not limited to the particular arrangements set out herein and instead extends to encompass all arrangements, and modifications and alterations thereto, which fall within the scope of the appended claims.

For example, in an envisaged arrangement the apparatus may be supplied to a patient with the guard and bag pre-coupled to one another, for example by welding or adhering the guard to the bag. In another envisaged arrangement, the guard and bag could be integrated into a single component, with the guard comprising a flap that extends beyond the periphery of the bag—the flap being provided with an adhesive strip for forming a seal against the patient's skin.

In yet another envisaged arrangement, the cover may comprise three discrete components, one for adhering to the patient so that it covers (and adheres to) an upper (typically generally horizontal) edge of the bag, and the other two components for adhering to the patient so that they cover (and adhere to) each lateral (typically generally vertical) side of the bag.

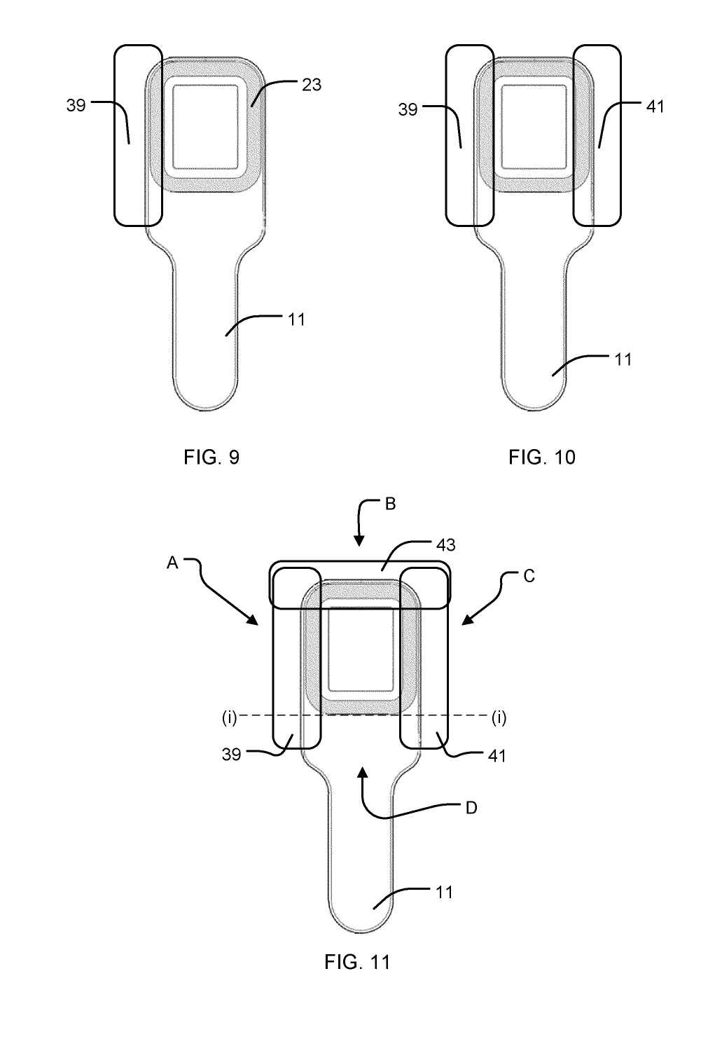

FIG. 9 illustrates a bag 11, in use, to which a first cover component 39 has been adhered (the or each catheter lumen being omitted from the drawing for clarity). The first cover component 39 is adhered to a patient's body and to the left-side generally vertical edge of the bag 11. In the arrangement shown in FIG. 9 the first cover 39 extends along the entirety of, and thereby covers, the left-side vertical portion of the adhesive 23.

FIG. 10 illustrates the bag 11 in FIG. 9 after a second cover component 41 has been adhered to the bag 11. The second cover component 41 is adhered to the patient's body and to the right-side generally vertical edge of the bag. In the arrangement shown in FIG. 10 the second cover component 41 extends along the entirety of, and thereby covers, the right-side vertical portion of the adhesive 23.

FIG. 11 illustrates the bag 11 in FIG. 10 after a third cover component 43 has been adhered to the bag 11. The third cover component 43 is adhered partially to the patient's body, partially to the upper generally horizontal edge of the bag 11, and partially to the first and second cover components 39, 41. In the arrangement shown in FIG. 11 the third cover component 43 extends along the entirety of, and thereby covers, the upper horizontal portion of the adhesive 23. The third cover component 43 also partially covers (at least the uppermost edges) of the first and second cover components 39, 41.

As with the arrangement illustrated in FIG. 4 it will be appreciated that the apparatus illustrated in FIG. 11 provides two seals against sideways water ingress (i.e in directions A & C), and downwards water ingress (i.e. in direction B). The apparatus only provides a single seal against water ingress in an upwards direction (i.e. in direction D), but as water flows downwards from a shower head, it is anticipated that a single seal will be more than adequate to deal with any water that might inadvertently splash upwards whilst the patient is showering.

Furthermore, as with the arrangement shown in FIG. 4 it will be appreciated that the first and second cover components 39, 41 need not extend downwardly beyond the adhesive strip 23 on the bag 11 (as shown in FIG. 11). The first and second cover components 39, 41 could instead terminate at a higher point (i) in the vicinity of the adhesive strip 23 on the bag. Whilst it is particularly preferred for the first and second cover components 39, 41 to at least extend to a point below a catheter exit site (not shown in FIG. 11 to enhance clarity of the drawing), it will be appreciated by persons skilled in the art that the first and second cover components 39, 41 may extend any length along the respective side edges of the bag 11 because this would still provide an arrangement that avoided many of the problems (in particular the aforementioned pooling problem) associated with the devices depicted in FIGS. 1 and 2 of the drawings.

Lastly, it should also be noted that whilst the accompanying claims set out particular combinations of features described herein, the scope of the present invention is not limited to the particular combinations hereafter claimed, but instead extends to encompass any combination of features herein disclosed. In particular it should be remembered that features of any one embodiment may be combined, at will, with any of the other embodiments disclosed herein.

1. Apparatus for shielding a catheter from contact with water whilst showering, the apparatus comprising:
a first shield component having a first part that is configured to form a first seal around an exit aperture from which a catheter exits the patient's body, said first part defining an internal volume that opens to an internal volume of a second part that is configured to be suitable for receiving a distal part of said catheter; and
a second shield component for adhering to the patient's body around at least part of the periphery of the first part of said first shield component so as to provide a second seal against water contact with said exit aperture;
wherein said first and second shield components cooperate, when the apparatus is worn by a patient, to resist water contact with said exit aperture without obstructing access to an area of the patient's skin beneath said second part of said first shield component.

2. The apparatus of claim 1, wherein the second shield component is configured to at least partially cover the first part of said first shield component.

3. The apparatus of claim 1, wherein the second shield component is provided with adhesive for forming said second seal.

4. The apparatus of claim 3, wherein the adhesive provided on said second shield component extends along at least one peripheral side of said second shield component.

5. The apparatus of claim 4, wherein the adhesive extends along more than one peripheral side of said second shield component.

6. The apparatus of claim 5, wherein the adhesive provided on said second shield component is generally C-shaped.

7. The apparatus of claim 6, wherein the first seal is located wholly within the C-shaped adhesive on said second shield component when the second component is fitted over the first shield component.

8. The apparatus of claim 1, wherein the first and second shield components are capable of being coupled to one another.

9. The apparatus of claim 8, wherein the first shield component and/or the second shield component further comprises an additional body of adhesive for fixably coupling the first and second shield components together.

10. The apparatus of claim 1, wherein the first part of said first shield comprises an entry aperture for receiving said catheter.

11. The apparatus of claim 10, wherein said first shield component comprises a liner accommodated within said aperture.

12. The apparatus of claim 1, wherein substantially all of one face of said second shield component is covered with adhesive.

13. The apparatus of claim 1, wherein said second part of said first component is narrower than said first part of said first component.

14. The apparatus of any of claim 1, wherein the second shield component comprises a plurality of discrete cover components.

15. (canceled)

16. The apparatus of claim 14, wherein the second shield component comprises first, second and third discrete cover components.

17. The apparatus of claim 16, wherein the first, second and third cover components are each configured to be coupled to the body of a patient and to the first part of said first shield component.

18. The apparatus of claim 17, wherein the first, second and third shield components are configured to extend along respective edges of the first part of the first shield component when coupled thereto.

19. The apparatus of claim 18, wherein the third cover component is configured to partially cover the first cover component and the second cover component when the first, second and third cover components are coupled to the first part of the first shield component.

20. Apparatus for shielding a catheter installed in a patient's body from contact with water whilst the patient is showering, the apparatus comprising:
a first shield component having a first part that is configured to form a seal around an exit aperture from which the catheter exits the patient's body, said first part defining an internal volume that opens to an internal volume of a bag-like second part of said first shield component, said second part of said first shield component being configured to enclose a part of said catheter distal from said patient; and
a second shield component configured to be adhered to the patient's body so that said second shield component overlaps and covers at least part of the periphery of the first part of said first shield component;
wherein said first and second shield components co-operate, when the apparatus is adhered to a patient, to provide two seals that each reduce the likelihood of water from a shower from contacting the exit aperture in the patient's body.

21. Apparatus for shielding a catheter in a patient's body from contact with water whilst the patient is showering, wherein said catheter extends from an exit aperture in said patient's body to at least one catheter tail distal from said exit aperture; the apparatus comprising:
a first shield component having a first part that is sealable around said exit aperture from which the catheter exits the patient's body, said first part defining an internal volume that opens to an internal volume of a bag-like second part of said first shield component, said second part of said first shield component being configured to enclose said at least one catheter tail; and
a second shield component separate from said first shield component, wherein said second shield component is configured to be adhered to the patient's body so that said second shield component overlaps and covers substantially all of the first part of said first shield component;
wherein said first and second shield components co-operate, when the apparatus is adhered to a patient, to reduce the likelihood of water from a shower from contacting the exit aperture in the patient's body without obstructing access to an area of the patient's skin beneath said second part of said first shield component.
